(19) United States
(12) Patent Application Publication
(10) Pub. No.: US 2021/0069033 A1
(43) Pub. Date: Mar. 11, 2021

(54) TAMPON STORAGE KEYCHAIN

(71) Applicant: Katherine Marie, Brooklyn, NY (US)

(72) Inventor: Katherine Marie, Brooklyn, NY (US)

(21) Appl. No.: 17/015,118

(22) Filed: Sep. 9, 2020

Related U.S. Application Data
(60) Provisional application No. 62/922,938, filed on Sep. 9, 2019.

(51) Int. Cl. A61F 13/551; A61F 13/20

(52) U.S. Cl. CPC A61F 13/55175; A61F 13/20

(57) ABSTRACT

A tampon storage container, comprising a hollow cylindrical tube having a closed bottom and an open top, defining a cylindrical tampon storage volume as a storage container body; a lid configured to be removably coupled to the open top of the body; and an attachment disposed on the tube cap, configured to be removably coupled to a portable object carried by a user.

This application claims priority of U.S. Provisional Application No. 62/922,938 entitled Tampon Keychain filed Sep. 9, 2019, which is hereby incorporated by reference in its entirety as if fully set forth herein.

### BACKGROUND

Although women of child-bearing age typically experience menstruation on a periodic cycle, unexpected irregular menstruation or feminine spotting can occur at virtually any time. If a feminine hygiene product is unavailable, inconvenient and undesirable consequences may occur such as clothing being stained, soiling of seats and other surfaces, and the like.

### SUMMARY

An unobtrusive, convenient, and pleasant-looking container in which a tampon may be carried, for use especially when unexpected menstruation or spotting occurs while away from home. The container may be removably coupled to a keychain or other object that is commonly carried when away from home. The tampon can be quickly and easily removed from the container for use when needed.

It is to be understood that both the foregoing general description and the following detailed description are exemplary and explanatory and are intended to provide

### BRIEF DESCRIPTION OF THE DRAWINGS

The accompanying drawings are included to provide a further understanding of the invention, and are incorporated in and constitute a part of this specification. The drawings illustrate disclosed embodiments and/or aspects and, together with the description, serve to explain the principles of the invention, the scope of which is determined by the claims.

In the drawings:

FIGS. 1A and 1B are top perspective and side views, respectively, of an exemplary embodiment of a tampon storage container.

FIGS. 2A, 2B, and 2C are perspective view of an exemplary embodiment of a container body and lid, an exploded view of the container and lid, and a sectional view of the container with tampon stored inside.

FIG. 3 is a side view of another embodiment, in accordance with the disclosure.

FIG. 4 is a view of an exemplary finished embodiment.

### DETAILED DESCRIPTION

It is to be understood that the figures and descriptions provided herein may have been simplified to illustrate aspects that are relevant for a clear understanding of the herein described processes, machines, manufactures, and/or compositions of matter, while eliminating, for the purpose of clarity, other aspects that may be found in typical devices, systems, and methods. Those of ordinary skill in the pertinent art may recognize that other elements and/or steps may be desirable and/or necessary to implement the devices, systems, and methods described herein. Because such elements and steps are well known in the art, and because they do not facilitate a better understanding of the present disclosure, a discussion of such elements and steps may not be provided herein. However, the present disclosure is deemed to inherently include all such elements, variations, and modifications to the described aspects that would be known to those of ordinary skill in the pertinent art.

It will be readily understood that the components of the present invention, as generally described and illustrated in the figures herein, may be realized in a variety of different configurations. Thus, the following detailed description of the embodiments of a method, apparatus, and system, as represented in the attached figures, is not intended to limit the scope of the invention as claimed, but is merely representative of selected illustrative embodiments of the invention. The usage of the phrases “example embodiments”, “some embodiments”, or other similar language, throughout this specification refers to the fact that a particular feature, structure, or characteristic described in connection with the embodiment may be included in at least one embodiment of the present invention, and do not necessarily all refer to the same group of embodiments.

One or more embodiments will now be described. These embodiments are provided as teaching examples and should not be interpreted to limit the scope of the invention. Although specific details of the embodiments are presented, the embodiments may be modified by changing, supplementing, or eliminating many of these details.

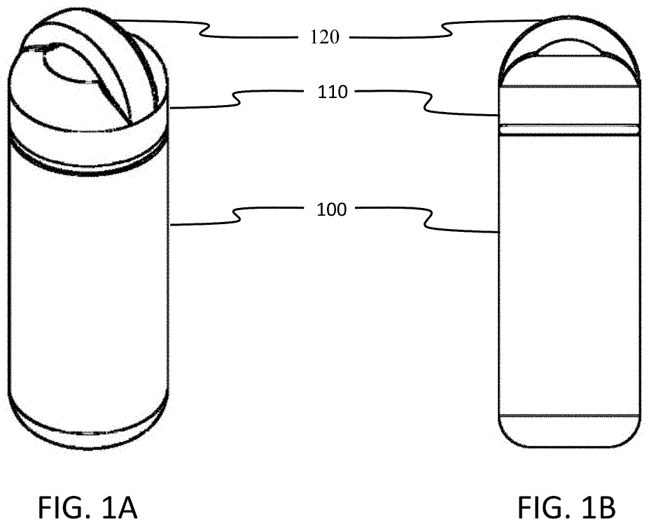

FIGS. 1A and 1B show a top perspective view and a side view, respectively, of an embodiment of a container for storing a tampon. The embodiment comprises a generally right circular cylindrical hollow body 100, enclosed at a bottom end and open at a top end thereof, thereby forming a tampon storage volume. A lid 110 may be removably coupled to the opening at the top end of the body 100. The lid 110 comprises a fastener 120, configured to be removably coupled to one or more objects carried by a user, such as a plurality of keys as a keychain. The fastener 120 may also be removably coupled to other objects, such as a handbag, a belt loop, or the like. The lid 110 may have an outer diameter substantially equal to that of the body, although this is not required.

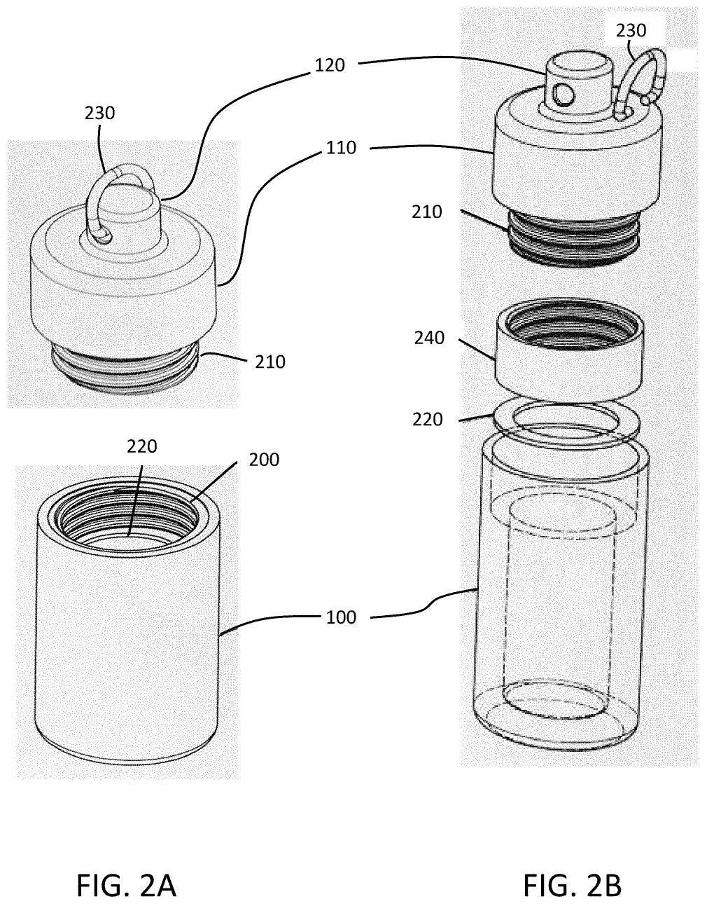

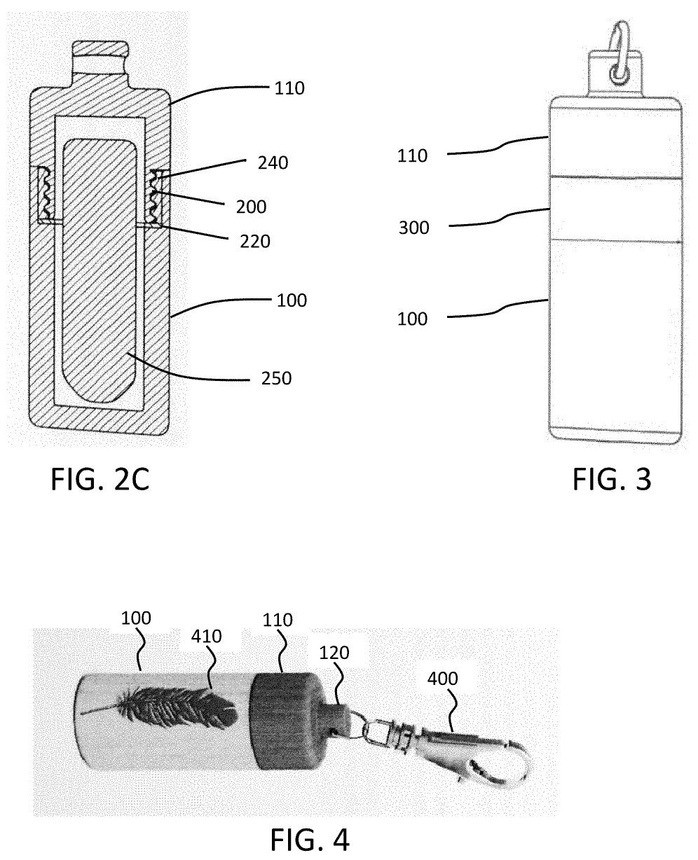

FIG. 2A is a perspective view of the body 100 and lid 110 of another exemplary embodiment. As shown, the lid 110 is removably coupled to the body 100 by screwing matching threads together, although other types of temporary joints may alternatively be used. For example, a fitting may be used in which an extension with a smooth side surface disposed on the bottom of the lid 110, slides into a matching hole with a smooth side surface disposed at the top of body 100, wherein the lid may be pushed onto the body, and the body and lid are held together by friction between the smooth side surfaces where they overlap (not shown). Moreover, a small ridge may extend around the circumference of the smooth side surface on one or both of the body 100 and the lid 110, that fits into a matching groove on the other of the body 100 and the lid 110, to more securely hold the body 100 and the lid 110 together (not shown). Referring again to FIG. 2A, threads 200 on the inside surface of the top of body 100 may screw together with matching threads 210 on the outside surface of an extension disposed at the bottom of the lid 110 to hold the body and the lid together. A washer 220 or the like may be inserted between the body 100 and the lid 110 to provide a tight seal between them.

Moreover, a fastener 120 is disposed on the top of the lid 110, configured to be removably coupled to one or more objects carried by a user. As shown, the fastener includes a staple 230 with bent ends inserted into holes in the fastener body, to which a caribiner may be attached, as will be shown.

FIG. 2B is a perspective view of a container 100 with lid 110, similar to that of FIG. 2A, wherein the container is shown as an exploded view showing various components before assembly. As shown, the bent ends of the staple 230 (to which a caribiner may be attached) are not yet inserted into the holes in the fastener 120 body. Further, the body 100 of this embodiment comprise a hollow cylindrical insert 240. The cylindrical insert 240 may have a smooth outside surface that slides into a matching hole with a smooth surface disposed at the top of body 100. The insert 240 may be pushed onto the body, and the body and insert are held together by friction between the smooth side surfaces where they overlap. After the insert 240 is inserted into the hole at the top of body 100, as described previously the threads 200 on the inside surface of the insert 240 may screw together with the matching threads 210 on the outside surface of the extension disposed at the bottom of the lid 110 to hold the body and the lid together. Moreover, washer 220 may be placed into the hole at the top of body 100 before the insert is pushed into the hole, to provide the tight seal between the body 100 and the lid 110 when they are screwed together. FIG. 2C shows the assembled body 100, including washer 220 and insert 240 with threads 200, with the body and lid 110 screwed coupled together. A tampon 250 has been placed inside the container for storage.

FIG. 3 shows an embodiment in which a hollow cylindrical extension 300 may be inserted between body 100 and lid 110. Preferably, the extension 300 may be formed with an inside diameter and outside diameter substantially the same as those of the body 100. Further, threads in the inside surface at the top of extension 300 match the threads disposed at the bottom of lid 110, and threads in the outside surface at the bottom of extension 300 match the threads disposed at the top of body 100. Accordingly, the extension serves to make the storage space in the container longer than it would be with the body and the lid screwed together directly. Thereby, a longer tampon can be stored in the container using the extension 300 than can be stored therein without the extension 300.

FIG. 4 shows an embodiment wherein body 100 and lid 110 have a different finish, either because they are made of different materials, or they are of the same material with different paint, stain, or other finish. For example, the lid 110 may be made of stained wood, while the body 100 may be made of painted wood. Of course, the container may be made of one or more other suitable materials, such as metal or plastic.

The embodiment in FIG. 4 is shown with a carabiner 400 coupled to the staple with bent ends inserted in holes at the top of the faster body 120. The carabiner may then be coupled to a key ring, belt loop, or other object carried by a user. In addition, a logo 410 may be printed or painted on any appropriate part of the container. As shown, an image of a feather 410 has been stenciled as a logo or decoration on the side of the container body 100.

Although the invention has been described and illustrated in exemplary forms with a certain degree of particularity, it is noted that the description and illustrations have been made by way of example only. Numerous changes in the details of construction, combination, and arrangement of parts and steps may be made without deviating from the scope of the invention. Accordingly, such changes are understood to be inherent in the disclosure. The invention is not limited except by the appended claims and the elements explicitly recited therein. The scope of the claims should be construed as broadly as the prior art will permit. It should also be noted that all elements of all of the claims may be combined with each other in any possible combination, even if the combinations have not been expressly claimed.

What is claimed is:

1. A tampon storage container, comprising:
a hollow cylindrical tube having a closed bottom and an open top, defining a cylindrical tampon storage volume as a storage container body;
a lid configured to be removably coupled to the open top of the body; and
an attachment disposed on the tube cap, configured to be removably coupled to a portable object carried by a user.

2. The tampon storage container of claim 1, wherein the attachment disposed on the tube cap is a caribiner coupled to the lid.

3. The tampon storage container of claim 1, further comprising a graphic design element affixed to an outside surface of the storage container, as one of a logo and a decoration.

4. The tampon storage container of claim 1, further comprising an extender having the same inside diameter, outside diameter, and lid coupling elements as the storage container body, for elongating the tampon storage volume.

5. The tampon storage container of claim 1, further comprising threads disposed at the top of the container body, and matching threads disposed at the bottom of the lid, wherein the lid is removably coupled to the open top of the body by screwing together the matching threads on the bottom of the lid and the top of the body.

6. The tampon storage container of claim 1, further comprising a washer disposed between the body and the lid to form a tight seal therebetween.

7. The tampon storage container of claim 1, further comprising a threaded insert disposed in a hole at the top of the container body as the threads disposed at the top of the container body.
